(19) United States
(12) Patent Application Publication
(10) Pub. No.: US 2003/0018240 A1
(43) Pub. Date: Jan. 23, 2003

(54) Chronic heart failure patient identification system

(76) Inventors: Gary A. Goetzke, St. Paul, MN (US); Malcolm E. Reid, St. Paul, MN (US); Angeline M. Carlson, Eden Prairie, MN (US)

Correspondence Address: MEDTRONIC, INC., 710 MEDTRONIC PARKWAY NE, MS-LC340, MINNEAPOLIS, MN, 55432-5604, US

(21) Appl. No.: 09/844,883

(22) Filed: Apr. 27, 2001

(51) Int. Cl. A61B 5/00

(52) U.S. Cl. 600/300

(57) ABSTRACT

Potential chronic heart failure patients are identified in a population such as an employer or medical care payer database using a method or computer software product to improve accuracy in identifying potential chronic heart failure patients, decrease the time required to identify potential chronic heart failure patient increasing opportunities for early intervention, identify selected potential chronic heart failure patients based upon preference of stakeholders, and many other benefits. Desired patient indicia including direct medical indicia, indirect medical indicia, and non-medical indicia are selected to serve as independent variables. At least one chronic heart failure indication is selected to serve as a dependent variable. A chronic heart failure model is created using the patient indicia and the chronic heart failure indication. The chronic heart failure model is applied to the population and potential chronic heart failure patients are identified by selecting individuals from the population that conform to the chronic heart failure model. Many different embodiments of the chronic heart failure patient identification system method and software product are possible.

### CROSS REFERENCE

This application is also related to the following co-pending applications entitled “Chronic Pain Patient Risk Stratification System” by inventors Goetzke et al. (attorney docket number P9640.00).

### BACKGROUND OF THE INVENTION

This disclosure relates to a medical information system and more specifically to a chronic heart failure patient identification computer program and method.

Although medical treatment of acute injuries and illnesses have improved significantly over the past few decades, chronic disease remains by far the greatest cause of mortality, diminished quality of life, and increased healthcare expenditures. Approximately 80% of healthcare costs are spent on the treatment of chronic disease, much of it on unnecessary hospitalizations, inappropriate medical interventions, and poor overall coordination of care. This is true because chronic diseases are commonly treated but quite frequently not appropriately managed. The bulk of these expenses are spent on cardiovascular disease, cancer, diabetes, AIDS, orthopedic and spinal disease, arthritis, and the full range of neurological diseases. In countries with an aging population, the prevalence of chronic disease will increase dramatically, further accentuating the need for better chronic care.

Historically chronic disease has often been considered part of normal aging with little attention given to prevention, precise diagnosis and fully coordinated, long-term treatment. This view of chronic disease manifests itself with relatively late-stage treatments conducted as a series of acute interventions after a critical episode. Treatments after a critical episode are typically more invasive, expensive, and less effective at restoring an individual to a full health than treatments that could be given prior to episode if only the chronic disease risk or symptoms had been more accurately diagnosed. The medical profession's focus on late-stage treatment of chronic disease after a series of acute interventions has been influenced by the compartmentalization of medical specialties around acute diseases that often do not provide optimal treatment for chronic diseases. The medical profession's lack of attention to chronic disease has also been slow to change because of the largely passive role payers, employers, health care policy makers and patients have played in the past.

The medical profession's perspective on chronic disease is changing through increased knowledge and access to better data and more meaningful information that are changing historical views. Adding momentum to the medical profession's understanding of chronic disease is the empowerment of payers and patients. Payers are pressuring the medical profession to control the high cost of chronic disease treatment. Payers understand that chronic disease costs can often be substantially reduces through a better understanding of chronic disease risks, early and accurate diagnosis, appropriate intervention, and fully coordinated, long-term care. Patients are empowered with informational technologies to ask questions, understand disease risks and symptoms, understand alternatives including complimentary therapies, and seek treatments that improve both length and quality of life. With the change in focus on chronic disease, there is recognition that the following chronic diseases that are not effectively managed: cancer, cardiovascular diseases, neurological diseases, musculo-sketetal diseases, diabetes, gastro-intestinal diseases, and chronic pain. The chronic heart failure population is among the most difficult to identify in the early stages of the disease. Yet, early stage identification will allow the treating physician to make the most significant impact on the patient's health care outcome. It will also provide the patient an opportunity to become involved in the management of their health condition. And, because of the importance of proper management of medical and non-medical risk factors, patient involvement is critical to successful treatment of heart failure.

Chronic heart failure results for many reasons. All people, over time, loose a certain level of heart function. In addition, a person's heart can become stressed by a wide variety of factors, including their lifestyle choices such smoking drinking, over eating or poor dietary choices, viral infections or high blood pressure. A person can be born with a congenital defect that could eventually lead to heart failure, or a person could have another medical condition such as anemia, high blood pressure, hyperthyroidism and a sever lung condition, that can overly stress the heart. These stresses, can force the heart to work too hard or directly damage the heart. In both cases, the result can lead to heart failure.

Previous clinical efforts have not been effective in identifying patients who are at risk for chronic disease, patients that have undetected chronic disease, and patients that have been misdiagnosed for a condition other than their actual chronic disease.

Previous clinical efforts have been particularly ineffective in identifying patients that are at risk for early stage chronic heart failure, linking indirect medical indicia to a heart failure condition, identifying patients who have undetected chronic heart failure, and identifying patients who have been misdiagnosed for a condition other than their actual chronic heart failure.

For the foregoing reasons, there is a need for a chronic disease patient identification system that permits earlier and more effective intervention to treat chronic disease to improve patient health, reduce costs, and provide additional benefits.

### SUMMARY OF THE INVENTION

The chronic heart failure patient identification system can be a method or computer software product that identifies individuals at risk for a chronic heart failure indication in a population. Desired patient indicia including direct medical indicia, indirect medical indicia, and non-medical indicia are selected to serve as independent variables. At least one heart failure indication or condition is selected to serve as a dependent variable. A chronic heart failure model is created using the patient indicia and the chronic heart failure condition. The chronic heart failure model is applied to the population and potential chronic heart failure patients are identified by selecting individuals from the population that conform to the chronic heart failure model. Some embodiments can include establishing selection preferences that specify patient characteristics desired to be selected by a stakeholder such as a patient, primary care physician, specialist physician, employer, or payer. The selection preferences are calculated with each potential chronic heart failure patient's mathematical expression to identify relationships between the selection preferences and each potential chronic heart failure patient's mathematical expression. Each potential chronic heart failure patient is categorized based upon the relationships between the selection preferences and each potential chronic heart failure patient's mathematical expression. Some embodiments can include sensitivity analysis to improve accuracy of the chronic heart failure patient identification system. The sensitivity analysis includes comparing the identified chronic heart failure patients with outside patient indicia to create a patient error list. An error assessment model is applied to the patient error list to identify the non-corresponding patient indicia that contributed to the errors. A sensitivity analysis model is applied to the non-corresponding to the non-corresponding patient indicia to identify potential patient indicia changes to reduce errors in identifying chronic heart failure patients. At least one patient indicia change is selected from the potential patient indicia changes to apply to the patient indicia to modify the patient indicia. Many different embodiments of the chronic heart failure patient identification system method and software product are possible.

### BRIEF DESCRIPTION OF THE DRAWINGS

FIG. 1 shows a block diagram of a chronic heart failure patient management system embodiment;

FIG. 2 shows a block diagram of a chronic heart failure patient identification system embodiment;

FIG. 3 shows another block diagram of a chronic heart failure patient identification system embodiment;

FIG. 4 shows a more detailed block diagram of a chronic heart failure patient identification system embodiment;

FIG. 5 shows a table of direct medical indicia prophetic example embodiment;

FIG. 6 shows a table of indirect medical indicia prophetic example embodiment;

FIG. 7 shows a table of non-medical indicia prophetic example embodiment;

FIG. 8 shows a block diagram of a chronic heart failure patient data preparation embodiment;

FIG. 9 shows a block diagram of a chronic heart failure model development embodiment;

FIG. 10 shows a Chi-Square Automatic Interaction Detection (CHAID) analysis prophetic example embodiment;

FIG. 11 shows a logistics table prophetic example embodiment;

FIG. 12 shows a block diagram of applying preferences to a patient mathematical expression;

FIG. 13 shows a block diagram of a sensitivity analysis chronic heart failure patient identification system embodiment; and,

FIG. 14 shows a more detailed block diagram of a sensitivity analysis chronic heart failure patient identification system embodiment.

### DETAILED DESCRIPTION OF THE PREFERRED EMBODIMENTS

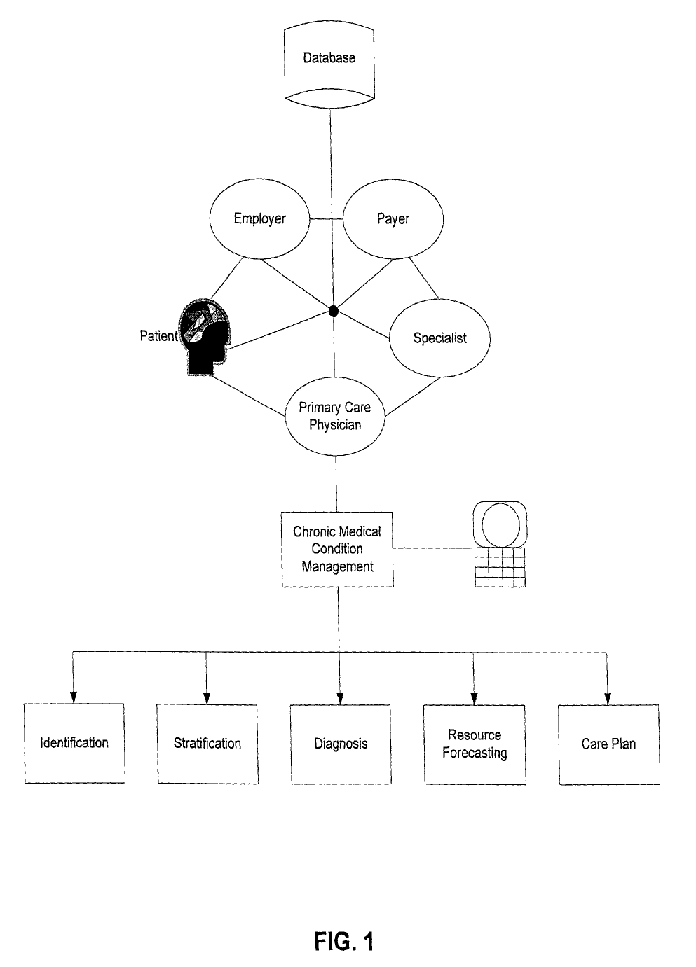

FIG. 1 shows a block diagram of a chronic medical condition management system embodiment and some elements of its operating environment. The chronic medical condition management system integrates the requirements and interests of at least five stakeholders include the patient, employer, payer, medical specialist, primary care physician, and the like. Other parties can also be added such as federal government, state government, allied health care professionals such as dietitians, nurse specialists, health educators, and the like. The chronic medical condition management system can operate on data controlled by each stakeholder and on data contained in a common database. The management system can be operated on a variety of computer systems depending upon the complexity of the management system such as a personal computer, minicomputer, mainframe computer, super computer, and the like. The management system can contain one or more components such as a chronic heart failure patient identification system, chronic heart failure patient risk stratification system, chronic heart failure patient diagnosis system, chronic heart failure patient dynamic resource forecaster, and chronic heart failure patient dynamic care plan. All the stakeholders typically desire a health care delivery process that provides appropriate and efficacious care in a cost effective manner, but this desire takes on different meanings depending upon the perspective of the stakeholder. These perspectives are built into the software in the form of categorization preferences, which will later be taken into consideration when making software-driven choices. Since each stakeholder can use system-generated data for different purposes, each stakeholder can have a customized view and access to the data. The system also profiles these data needs as data preferences, and data is provided in accordance with customized data requirement profiles. Following is a brief discussion of each stakeholder's interest.

Employers are typically interested in resource stewardship, maintaining a safe work environment for their workers, enhancing work force productivity, and the like. From an employer's perspective, a safe, healthy, and happy work force translates into improved worker productivity. For this reason many employers strive to understand and meet the basic health care needs of their work force but seek to do so in a cost effective manner. Employers are more engaged than ever in designing benefit packages for their employees. They will typically endorse efficacious, lowest cost treatments and particularly those designed to promptly return an injured employee to work. To make such benefit decisions, employers need data. Information relating cost benefit analysis and similar data that will allow them to compare therapies based upon clinical effectiveness and cost is very useful. Return to work data is also of critical importance. There is a host of other data points that employers would find useful, but which is data that is not typically collected or well understood. For example, employers would find it helpful to better understand the cost of patient compliance vs. non-compliance with specific treatment options. Information that could profile an employee to predict patient compliance, could be crucial to the decision making process. Also, work environment data, such as knowing whether injury patterns can be identified among a work force, could allow employers to develop targeted strategies to reduce or eliminate work place injuries.

Payers are typically interested in ensuring that clinically effective care is provided to health care members in a cost effective manner that provides a high level of reported patient satisfaction. The role of the payer is evolving with time, and in the future, payers will become more involved in population management for specific disease states. For this reason, payers will require epidemiological data. Payers desire to be more involved in educating their members on specific disease states, personalizing responses to match the specific needs of their members. Additionally, payers require clinical and economic data in a format that business leaders are accustomed to using in the decision making process. In short, payers are evolving their data collection practices to become more practical partners with employers, as both parties strive to tailor benefits to meet the needs of a defined population of employees.

Specialists are typically interested in having patients referred that are appropriate for the specialist scope of practice. Health care payers increasingly demand more rigorous proof of therapy value. The evidence is requested in the form of clinical, quality of life and economic outcome studies, claims-based retrospective studies, or economic models. Physicians are becoming more involved in the data collection, interpretation and reporting process, and it is quite common for them to develop their own data bank of information on patient outcomes. In addition, the specialist is typically a part of a care team, and the primary care physician usually acts as the gatekeeper of care. Depending upon the primary care physician's approach toward care delivery, the care team is either loosely coordinated or more actively coordinated, or sometime not at all coordinated. However, care coordination is becoming more and more a valued process, as payers and providers are realizing that a seamless and more efficient care process has a direct impact on therapy outcome and cost. For this reason, it is important for the entire team to communicate with each other and to adopt uniform processes for care delivery and outcome reporting. As patients become more actively engaged in the care delivery process, the specialist is also striving to evolve the communication relationship with their patients. Patients are becoming informed consumers of health care services, and specialists are responding by creating new means of communicating with patients. For example, it is quite common for specialists to have their own patient-focused website.

Primary care physicians are typically interested in making a proper diagnosis of their patients and making a proper decision on when a patient should be referred to a specialist. The data and communication needs of the primary care physician are similar to those of a specialist. Additionally, the primary care physician is finding it of practical value to have disease specific information readily available across a broad array of topics. Patients are asking questions that are more detailed about their condition, and often approach physicians with information they pulled from the web relating to a potential therapy or new drug that might be of potential treatment benefit. Being a generalist by training, the primary care physician often finds it useful to easily access clinical summaries, suggested treatment standards or other similar information that helps them decide how to initiate the management of a condition.

Patients are typically interested in participating in their health care, proper diagnosis of their medical condition, and effective treatment of their medical condition. They are seeking to better understand their medical condition and to become more actively informed in health care decision-making and more active participants in the treatment process. As more of the payment burden is shifted onto the patient, patients are becoming “care shoppers”, and therapy-specific economic data is more relevant to making an informed choice. Patients are also beginning to leverage web technology, using the web to get general disease information as well as to obtained more tailored information, programs or services that are personalized to their medical condition. The web is also being more frequently used as a means of communication between patients and their care providers, and is beginning to take the place of the telephone call and the physician office visit in the care delivery process. One component of the chronic heart failure patient management system is the chronic heart failure patient identification system.

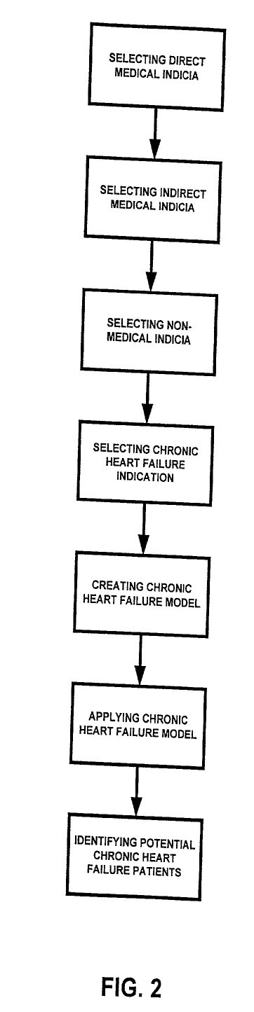

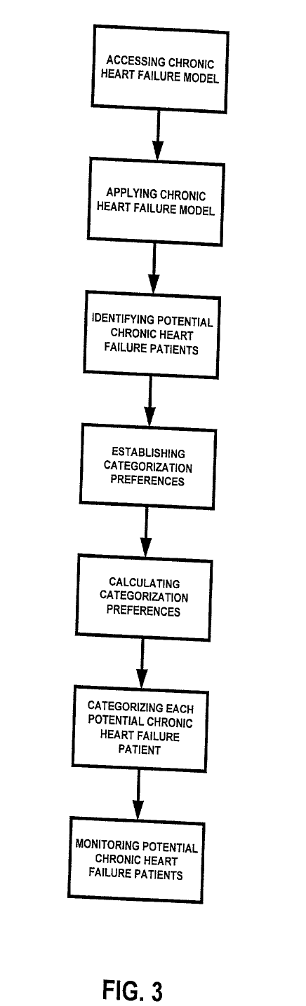

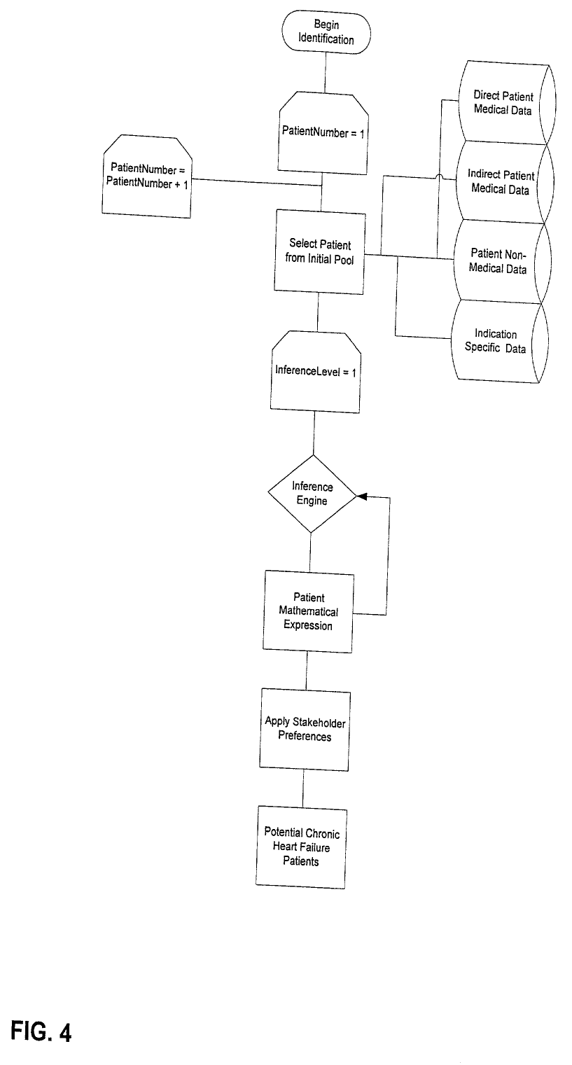

FIGS. 2 and 3 show block diagrams of chronic heart failure patient identification system embodiments, and FIG. 4 shows more detailed block diagram of a chronic heart failure patient identification system embodiment. The chronic heart failure patient identification system comprises the general elements of selecting patient indicia to evaluate, selecting a chronic heart failure indication, creating a chronic heart failure model using the patient indicia and the chronic heart failure indication, applying the chronic heart failure model to a population, and identifying patients at risk for chronic heart failure. Additionally, some embodiments can include accessing the chronic heart failure model, applying the chronic heart failure model, establishing categorization preferences for desired categories of potential chronic heart failure patients, calculating the categorization preferences with each potential chronic heart failure patient's mathematical expression to establish relationships, categorizing each potential chronic heart failure patient based upon these relationships, and monitoring the potential chronic heart failure patient. The patient indicia are selected from sources such as claims records, medical records, workers' compensation records, and employer records. The chronic heart failure model is applied to a population such as a payer database, employer database, primary care physician database, and the like.

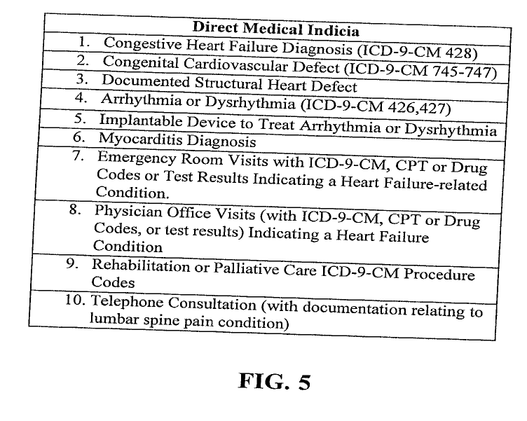

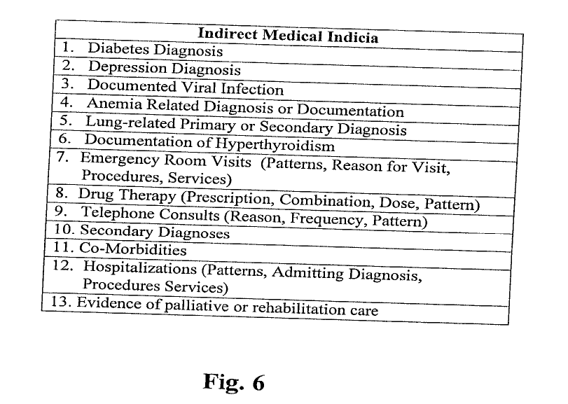

FIG. 5 shows a prophetic table of some direct medical indicia related to chronic heart failure, and FIG. 6 shows a prophetic table of some direct medical indicia in the form drug products. Although the indicia in FIGS. 5-6 are labeled direct medical indicia, under some circumstance certain of these direct indicia could also be classified as indirect indicia. The columns in FIGS. 5-6 and 8 are a gross simplification used to show how the chronic heart failure model could evaluate each indicium. Patient indicia would actually be included in the chronic heart failure model and applied to a broad population.

Direct medical indicia associated with chronic heart failure are selected to serve as independent variables for the chronic heart failure model. Direct medical indicia include information, recorded by a clinician, relating to a chronic heart failure indication of a patient. In addition to the direct medical indicia shown in FIGS. 5-6, direct medical indicia can also include indicia such as primary diagnosis, associated secondary diagnosis, co-morbidities, drug treatment regimen, telephone consultations with a clinician, palliative care, rehabilitative care, clinician office visits, emergency room visits, hospitalizations, and the like. Some direct medical indicia can be expressed as codes derived from nationally recognized coding systems such as International Classification of Diseases (ICD), American Medical Association Administrative Current Procedural Terminology (CPT); Healthcare Financing HCPCS), and National Drug Codes (NDC) shown in FIG. 5. Direct medical indicia are available from sources such as claims records, medical records, workers' compensation records, employer records, and the like. The importance of each of direct medical indicia is typically supported by the current body of chronic heart failure clinical literature, and can also be bolstered by expert medical opinion.

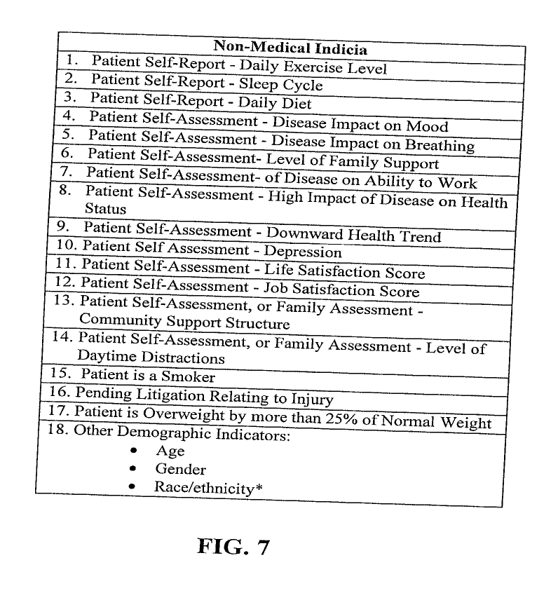

FIG. 7 shows a prophetic table of some indirect medical indicia. Indirect medical indicia associated with chronic heart failure are selected to serve as independent variables for the chronic heart failure model. Under some circumstances, the indirect medical indicia could be considered direct medical indicia. Indirect medical indicia include information recorded by a clinician relating to a patient's health condition but non-specific to the disease of chronic heart failure. Studies support the link between direct and indirect medical indicia in predicting the presence of chronic heart failure. Relevant indicia include such criteria as the patient's mental health status as indicated by a mental health ICD-9-CM diagnosis, as well patient's history of diabetes requiring hospitalization, emergency room visits or other related health care services.

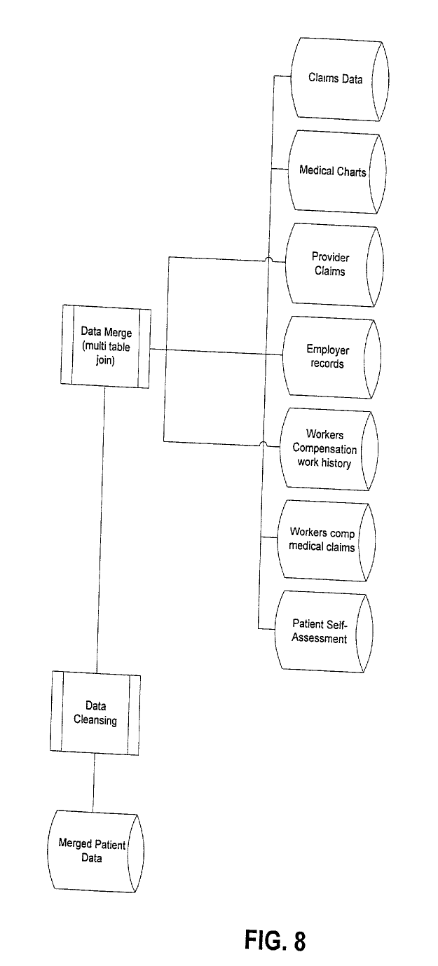

FIG. 8 shows a prophetic table of some non-medical indicia. Non-medical indicia associated with chronic heart failure are selected to serve as independent variables for the chronic heart failure model. Non-medical indicia include all indicia related to determining or predicting a person's health care status that is not medical indicia. Currently known non-medical indicia include life style behaviors such as alcohol consumption, smoking, caffeine intake, weight gain, sodium intake, self-assessed stress levels, life satisfaction measures, patient support structure from the family and the community at large, day time distractions, quality of marital relationship, and personality and psychological profiles. Additional non-medical indicia include demographic factors such as age, gender, and race or ethnicity. Non-medical risk indicia are mined from such sources as medical records; patient self-report documents; patient self-assessment surveys; employer databases; workers' compensation records; medical chart reviews; telephone interviews with patients, treating clinicians, and family members.

A chronic heart failure condition, also known as a chronic heart failure indication, is selected to serves as a dependent variable for the chronic heart failure model. Although the chronic heart failure model typically considers only one chronic heart failure indication dependent variable at a time, there can be chronic heart failure model embodiments that would consider multiple chronic heart failure indications simultaneously.

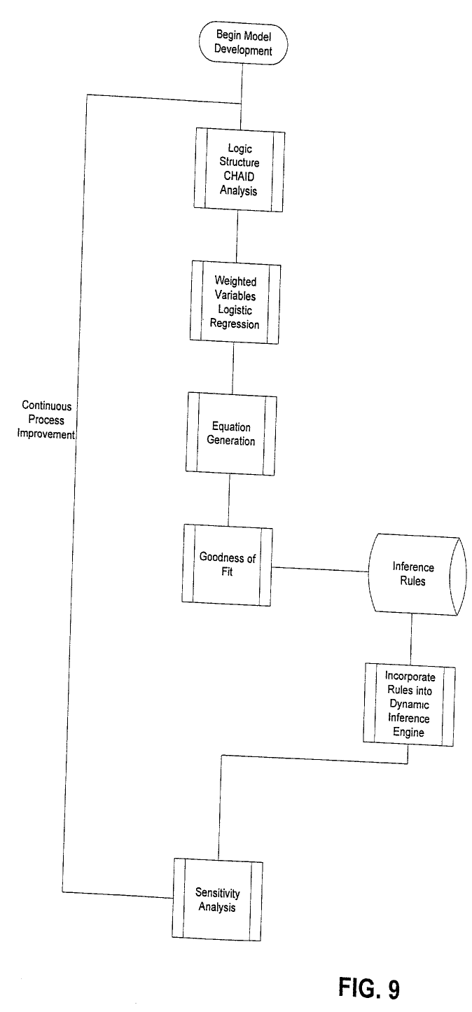

FIG. 9 shows a method for cleansing data such as patient indicia from potential data sources before the data is used in creating the chronic heart failure model. Often it is desirable to clean the data before the data is operated upon because data from various sources can have incompatible formats and data can contain errors. Data cleansing improves the reliability, accuracy and robustness of the chronic heart failure patient identification system.

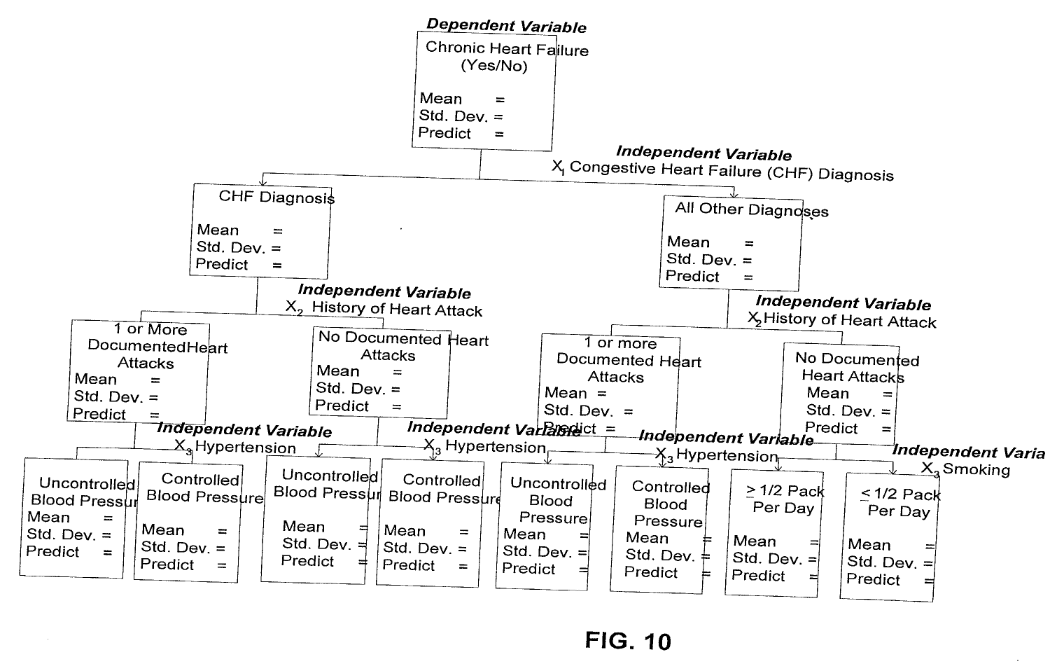

FIG. 10 shows a block diagram for creation of the chronic heart failure model in the form of a chronic heart failure inference engine embodiment. The chronic heart failure model comprises a logic structure, weighted variables, and equations. Some embodiments of the chronic heart failure model can include Hosmer-Lemeshow Goodness of Fit Analysis to evaluate the appropriateness of patient indicia, and monitoring patient indicia for changes that can be used to update the patient mathematical expression. The chronic heart failure inference engine can operate on at least fifty dependent variables, at least thirty independent variables, and at least fifty equations. The chronic heart failure model can be mathematically represented as follows: f(x)=b₀+b₁(X₁)+b₂(X₂)+b₃(X₃) . . . bᵢ(Xᵢ) where b₀ is a beta weight constant; b₁−bᵢ are the beta weights for each corresponding variable; X₁−Xᵢ are the significant variables identified from the model; and f(x) is the resultant measure of the characteristic of interest, i.e., chronic heart failure score. This chronic heart failure model equation creates a line that represents the minimized average for the dataset that is the line of prediction for the dataset.

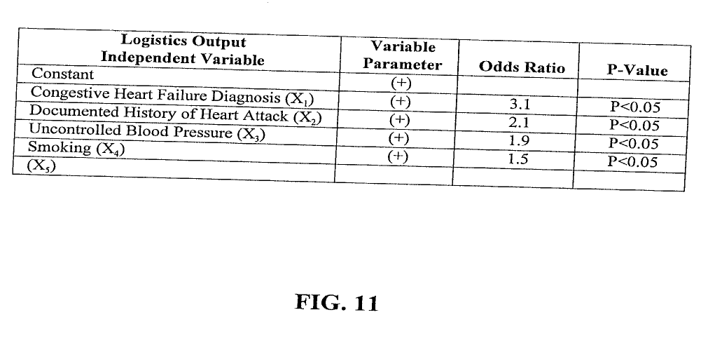

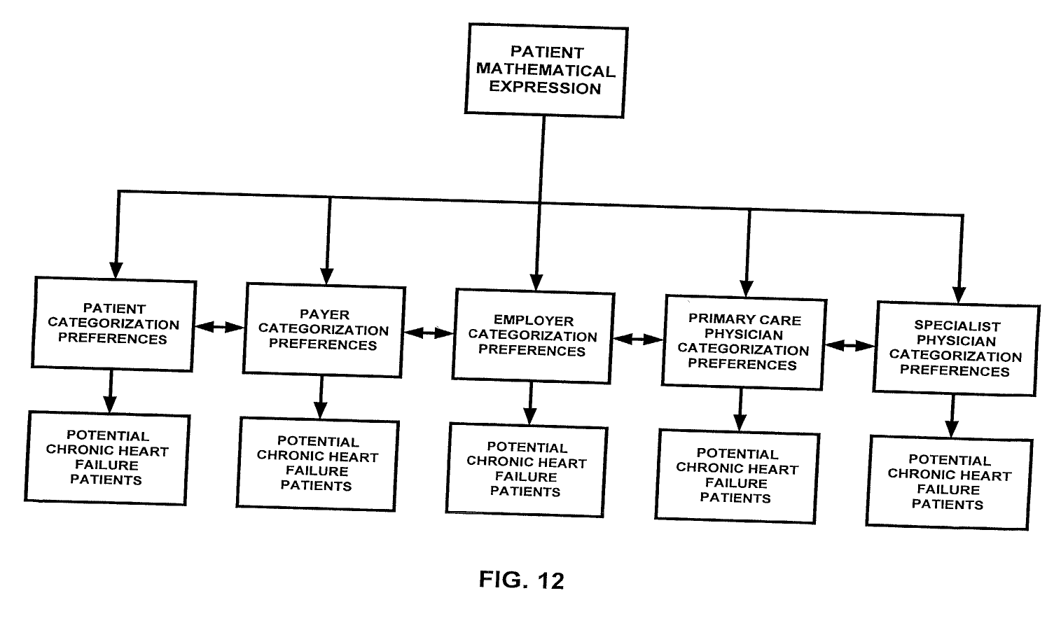

FIG. 11 shows a Chi-Square Automatic Interaction Detection (CHAID) analysis prophetic example embodiment, and FIG. 12 shows an analysis flow per indication prophetic example embodiment that was established by CHAID analysis. The logic structure used to establish relationships between a dependent variable and the independent variable can be developed using a statistical technique such as Chi-Square Automatic Interaction Detection (CHAID) analysis, CART analysis, and the like. The logic structure defines a logical decision process to progressively reach greater certainty about potential chronic heart failure patients. The logic structure can be evaluated using a statistical technique such as Hosmer-Lemeshow Goodness of Fit Analysis, and the like. CHAID is well known in the art, is an exploratory analysis executed to examine relationships that may exist between a dependent variable and multiple categorical variables that may interact with one another. It is predicated upon the supposition the necessary data is available, and that it is possible to distinguish, within a given data set, between two or more variables known to exist and known to be important.

CHAID is applied to the chronic heart failure construct in the following manner. Existing relevant information believed to be related to heart failure are culled from the clinical literature and bolstered by expert medical opinion, and a set of independent variables is identified based on current knowledge. As new clinical literature becomes available, the logic structure can be modified to include the new information. When the CHAID analysis is properly executed in a sequential fashion, the independent variables most clearly associated with the chronic heart failure measure will emerge.

The independent variables (predictors) are assessed to determine if splitting the sample based on these variables leads to statistically significant discrimination on the dependent measure. The most significant relationship defines the first split on the sample (called a branch or node). Then, for each group formed by the split, the remaining independent variables are assessed to determine which, if any, can further significantly discriminate on the subgroup. The end result (referred to as a terminal nodes) is a series of groups that are maximally different from one another on the dependent variable. At each step a statistical assessment is made to determine if a significant split into further subgroups can be made.

The length of the tree is the number of branches allowed to reach a terminal node. Tree length is set by the researcher and statistician based on decision rules. Based on the experience of the researcher, it has been determined that the model will continue branching until the variables found significant in differentiating the included population subsets establish nodes of N≦15 individuals. This analysis will identify variables for inclusion only if they are determined to be significant at the p<0.05 level. It is assumed that incorporating several different sources of non-medical risk data (Patient Survey, Employer records, etc.) will provide the necessary precision. An alternative to CHAID is Classification Adjusted Regression Tree (CART) analysis. However, CART does not have the same efficiency in creating the buckets of patients.

The CHAID technique presents certain advantages for this analysis. It provides a means of detecting patterns in what is a complicated set of data. The maximum amount of data is used because missing values can be incorporated into the analysis. The analysis allows for a nominal level of measurement on the dependent variable and the independent variables. Finally, the resultant model will emphasize strong results without over-capitalizing on chance occurrences because the many variables are considered at once in a step-wise fashion. Thus, CHAID is extremely useful in detecting data trends. In addition, it will allow formation of meaningful interaction terms, which will inform the estimation of probability in subsequent logistic regression analyses.

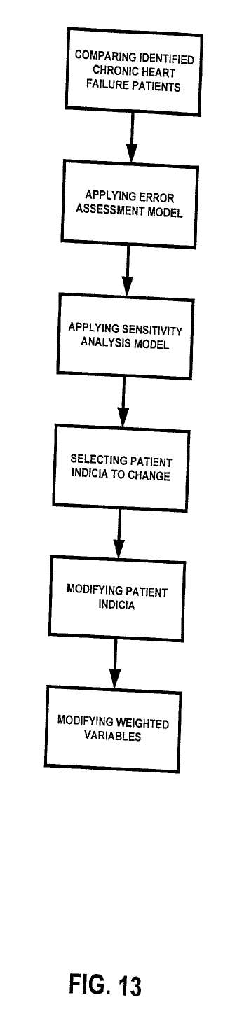

FIG. 13 shows a table with a prophetic logistic regression example. The weighted variables reflect greater relevance of certain direct medical indicia, indirect medical indicia, and non-medical indicia to the chronic pain indication. The weighted variables can be developed using a statistical technique to establish relationships between the dependent variable and independent variables such as logistic regression, discriminant analysis, and the like. Logistic regression is a form of statistical modeling appropriate for categorical outcome variables. The method examines the relationship between a categorical response, or dependent variable, and a set of explanatory, or independent variables. The results of logistic regression provide regression coefficients. The coefficients can be as simple as a single numerical value or as complex as an equation including known independent variables. After transformation, the regression coefficients can be interpreted as odds ratios describing the influence of various factors and the dependent variables. The logistic regression procedure provides odds ratios for independent variables as well as the significance level for each odds ratio. For example, the process could provide that people who smoke, are three times more likely to be chronic heart failure than people who do not. As with CHAID analysis, the many independent variables will be considered in a stepwise fashion, which allows for detection of the most explanatory of the variables. To be included in the logistic model variables must achieve a significance level of p<0.05.

Because the dependent variable has only two possible values (either chronic heart failure is present or it is not), it is not correct to assume that the variable would be normally distributed in a sample of individuals. By transforming the variable using a logistic function, the variable is made to appear closer to a normal distribution than would otherwise be the case (the assumption of a normal distribution being essential to the use of a linear statistical procedure). Taking into account the logistic transformation, the mathematical equation (or logistic function) that results from analysis takes the form:

$$Log \frac{p}{1 - p} = {b}_{0} + {b}_{1} ( {X}_{1} ) + {b}_{2} ( {X}_{2} ) + {b}_{3} ( {X}_{3} ) + {b}_{4} ( {X}_{4} ) … {b}_{i} {( {X}_{i} )}^{10}$$

where p in probability; b₀ is a beta weight constant; b₁−bᵢ is the beta weight for each corresponding variable; and X₁−Xᵢ are the significant variables identified from the model, e.g., X₁ can be congestive heart failure diagnosis, X₂ can be history of heart attack, and X₃ can be uncontrolled blood pressure. This logistic regression equation is further complicated by the potential interactions, described mathematically as follows: b(X₁·X₂). An alternative to Logistic Regression is Discriminant Analysis. Discriminant Analysis requires looking at extreme groups of patients. In order to find the most efficient group, the process requires a mix of extremes. Once logistic regression has been complete, equations can be generated.

Equations are generated to represent relationships between or among weighted variables to build a chronic heart failure inference engine. The chronic heart failure inference engine can operates on at east fifty dependent variables; at least thirty independent variables; and, at least fifty equations. The potential chronic heart failure patients are identified with a patient mathematical expression generated by the chronic heart failure inference engine operating on the patient indicia and the chronic heart failure indication. After a potential chronic heart failure patient is identified with a mathematical expression, that potential chronic heart failure patient's patient indicia can be monitored for relevant changes and the potential chronic heart failure patient's mathematical expression can be updated to reflect those changes. The computer will generate odds ratios and related significance levels as an output. Interpretation of results is a simple exercise of examining the sign (the direction of the parameter estimate), the value of the odds ratio, and it's significance level.

The number of equations generated can become quite large such as thousand and millions or equations associated with each chronic pain indication dependent variable, and currently there are 456 separate chronic pain indications. Due to the complexity and large number of equations, a computer is typically required to calculate the equations to produce a patient mathematical expression. A prophetic example of the number and complexity of equation generation follows. It is known that there are at least ______ different indications for heart failure. Assume a predictive model that accounts for each of these ______ dependent variables. Further assume that there are currently a total of 41 identified indicia for chronic heart failure, adding the direct and indirect medical and the non-medical indicia together (this number will grow as more is learned about chronic heart failure). If the model is developed out to the fourth level of independent variable (X₄) the calculation is as follows:

| Step | Equation Possibilities |
|---|---|
| 1 | Each indicia is considered individually: 41 total |
| | possibilities. |
| 2 | Each indicia is crossed with every other indicia for a |
| | two-way interaction calculation: 41 × 40 = 1640 total |
| | possibilities. |
| 3 | Each indicia is combined in a three-way interaction |
| | calculation: 41 × 40 × 39 = 63,960 total possibilities. |
| 4 | Each indicia is combined in a four-way interaction |
| | calculation: 41 × 40 × 39 × 38 = 2,430,480 total |
| | possibilities. |
| 5 | Total possibilities are added together; 2,496,121 total |
| | possibilities. |
| 6 | The model is run_(indications) different times |
| | with 2,496,121 possibilities for each indication. |

In addition to the complexity introduced by interaction terms, each time a new variable is identified and introduced into a model the logistic function must be regenerated. Any newly identified variable can dramatically affect the resultant model (the number of variables found to be significant, the value of the odds ratios found, and the directional relationship of the variables). New variables can be found to have significance when compared with previously tested variables and new variables can change the significance level of previously significant and non-significant variables or can change the way previous variables interact with either the new variable or previously identified variables. Thus as our knowledge of chronic heart failure expands, models generation must be revised, creating a dynamic knowledge opportunity limited only by our ability to identify and appropriately measure (both validly and reliably) additional variables and our ability to refine measurement of previously identified variables.

The potential complexity of chronic heart failure model can be seen from the following prophetic example. In the applied CHAID example, X₁ is “Congestive Heart Failure Diagnosis”. If it is discovered that X₁ is “Blood Pressure”, every other independent variable is potentially altered. This alteration includes order of importance, clusters of importance, and even relevance in terms of predictability. If X₁ becomes “Blood Pressure”, X₂ could likely become “Drug Treatment Regimen”. The weighted value of the cluster of these 2 indicia could be significantly higher than the cluster of the previous 2 indicia of “Congestive Heart Failure Diagnosis” and “Heart Attack”. The potential patient indicia, their importance and weight, alone and in combination with others can be immense.

The Hosmer-Lemeshow Goodness of Fit tests the models and determines whether the variables chosen for the model were the best possible. Once the logistic model is determined, the Hosmer-Lemenshow Chi-Square statistic is calculated to assess the goodness of fit of the model. A non-significant value indicates an adequate goodness of fit. If the Hosmer-Lemeshow analysis indicates that there is not a good fit, then the conclusion drawn is that there are variables other than those identified for model inclusion that might better explain the concept being investigated. This is an indication that further identification of variables and data sources for those variables must be determined.

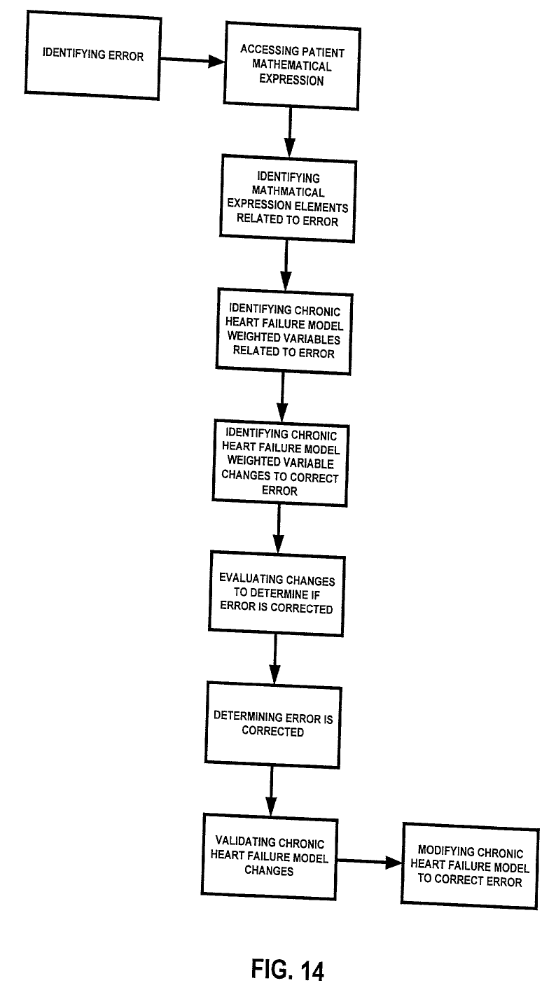

FIG. 14 shows a block diagram of applying categorization preferences to a patient mathematical expression embodiment. Potential chronic heart failure patient's can be categorized by first establishing categorization preferences that specify characteristics of patients desired to be categorized. The categorization preferences include patient categorization preferences, payer categorization preferences, employer categorization preferences, primary care physician categorization preferences, and specialist physician categorization preferences. The different stakeholder categorization preferences can be interrelated. For example, a payer categorization preference can include a potential chronic patient preference that might indicate whether the potential chronic pain patient would be compliance with a physical therapy regimen. Some examples of categorization preferences for a patient can include a desire to be notified of being a potential chronic heart failure patient even though the other stakeholders categorization preferences do not identify the patient as a potential chronic heart failure patient, a desire to not be notified of being a unless the other stakeholders would support treatment, a desire to not be notified under any circumstance of being a potential chronic heart failure patient. Some examples of categorization preferences for a payer include a desire to know if potential chronic heart failure patient reimbursement criteria are met and a desire to know whether the potential chronic heart failure patient special care program criteria are met. Some examples of categorization preferences for an employer can include a desire to know potential chronic heart failure patients who's job performance may be affected and potential chronic failure patients that can be efficiently treated. Some examples of categorization preferences for a primary care physician can include potential chronic heart failure patients who are suitable for treatment by the primary care physician and potential chronic heart failure patients that should be considered for referral to a specialist. Some examples of categorization preferences for a specialist physician can include potential chronic heart failure patients that are suitable for treatment by the specialist physician and potential chronic heart failure patients that should be considered for referral to a primary care physician.

The categorization preferences are calculated against each potential chronic heart failure patient's mathematical expression to identify relationships between the categorization preferences and each potential chronic heart failure patient's mathematical expression. Calculation of categorization preference can range from simple search and find algorithms to complex statistical models such a modified chronic heart failure model.

The software assigns an alphanumeric score for each patient identified under the rules of the inference engine. The number score, based upon a 0-100% rating, relates to the level of predictive confidence that an appropriate candidate has been identified. Patients with a confidence rating of ≧85% will be considered as potential chronic heart failure patients, and their names will be passed along to a primary care physician for an initial determination of program inclusion or exclusion. Patients with a lower than 35% rating will be excluded from further consideration. Patients with a score in the range of 35%-85% will be held in the system for up to one year, and the receipt of new information could alter their score upward or downward—triggering program inclusion or exclusion.

Letter designations represent heart failure etiology, as coded or described in the data, as well as any other rules-based, important identifying or describing characteristics or profiles of heart failure. For this reason, patients can receive more than one letter designation. For example, a patient suffering from an arrythmia would receive a “J” designation. If the patient also had an implantable defibrillator, he or she would also be designated as a “C”. It should be noted that a patient's letter designation is subject to change, based upon the receipt of additional relevant data. If no such feature can be identified from the data query, the letter P is assigned.

The following table lists the letter designations and explains the meaning of each designation. As system knowledge increases, this list will change through addition, deletion or modification.

Patient Rating System Table

| Designation | Definition |
|---|---|
| A | Congenital Defect |
| B | Structural Defect |
| C | Implantable Device |
| D | Myocarditis |
| E | Diabetes |
| F | Viral Infection |
| G | Lung Condition |
| H | Hyperthyroidism |
| I | High Blood Pressure |
| J | Arrythmia |
| K | Disrythmia |
| L | Heart Attack |
| M | Depression |
| N | Heavy Smoker |
| O | Addications |
| P | No Identified Characteristics |

Once potential chronic heart failure patients are selected, that patient's indicia can be monitored to detect changes that can affect their categorization. The selected potential chronic pain patient's direct medical indicia, indirect medical indicia, and non-medical indicia are monitored for changes and the patient's mathematical expression is updated based upon changes to the potential chronic pain patient's direct medical indicia, indirect medical indicia, and non-medical indicia.

FIG. 15 shows a block diagram of a method of sensitivity analysis of a chronic heart failure model embodiment, and FIG. 16 shows a block diagram of applying a sensitivity analysis model. The method can begin by comparing the identified potential chronic heart failure patients with outside diagnosed chronic heart failure patient data to create a patient error list. The outside diagnosed chronic heart failure patient data would typically include diagnosis information such as laboratory test results, patient survey data, physiologic measures, the specific chronic heart failure indication, and the like. Sources for outside diagnosed chronic heart failure patient data include medical claim data, medical charts, employer records, worker compensation records, and the like. The patient error list has an error assessment model applied to the patient error list to identify non-corresponding patient indicia that contributed to the errors. The non-corresponding patient indicia are typically the absence of one or more patient indicia or the inclusion of one or more extraneous patient indicia. The non-corresponding patient indicia has a sensitivity analysis model applied to the non-corresponding patient indicia to identify potential patient indicia changes to reduce errors in identifying chronic pain patients. Examples of potential patient indicia changes include the addition of one or more relevant indicia or the exclusion of one or more extraneous patient indicia. At least one patient indicia change is selected from the potential patient indicia changes for changing. Finally, the patient indicia are modified with at least one selected patient indicia change. The modified patient indicia typically improve accuracy of the method for new patients entered into the system because new patient indicia may be required. The modified patient can improve the accuracy of the method for patients currently entered into the system particularly if patient indicia are excluded.

The weighted variables of the chronic heart failure model can also be modified in a manner similar to the patient indicia. The sensitivity analysis model is applied to the weighted variables to identify potential weighted variable changes to reduce errors in identifying chronic heart failure patients. At least one weighted variable change is selected from the potential weighted variable changes to apply to the weighted variables. The weighed variables are modified to reflect greater or lesser relevance of patient indicia to reduce errors in identifying chronic heart failure patients.

### Prophetic Patient Examples

The following examples describes two individuals who, due to their unique combination of attendant direct medical, indirect medical and non-medical risk indicia, have been identified as potential chronic heart failure patients (hereinafter be referred to as Patient A and Patient B). The examples illustrate how indicia derived from data sources such as administrative and pharmaceutical medical claims data and patient self-report data, can be used to systematically identify and classify potential chronic heart failure patients. The identification process results in the patient being classified with a patient mathematical expression that could represent a complex description of the patient. The prophetic examples are used to illustrate just one of the many application of the chronic heart failure patient identification system and should not be read to limit application of the identification system.

Patient A has been identified through the application of patient identification software to a payer organization's freestanding administrative and pharmaceutical claims databases. The medical records indicate that Patient A is a 74-year-old male with a previous arrythmia diagnosis (within past 36 months) and a series of tests conducted in a catheter lab, confirming that diagnosis. Patient A received an implantable defibrillator to treat that condition within 30 days of the arrithmia diagnosis. The patient is overweight (250 pounds), and the medical record indicates that he has been experiencing recent changes in his lifestyle. A self-described home carpenter, plumber and electrician, he has recently been forced to limit his activities and now hires someone to do his home repair. Patient A complains of shortness of breath and no longer walks the three mile a day regiment he and his wife had engaged in for a number of years. He also has had difficulty in breathing while lying flat on his back. Patient A also complains of recent swelling in the feet and ankles. The combination of arrythmia diagnosis, implantable defibrillator and medical record notations relating to the patient's general medical condition, identify Patient A as a potential chronic heart failure patient.

Patient B is a 65-year-old male with an extended history of high blood pressure, well documented in the medical records and administrative and pharmaceutical claims data base. Patient B is a heavy smoker (2 packs per day, 40 years) as noted in the medical record by his physician and is also over-weight (220 pounds, 5′8″ tall). He has had a chest X-ray within the previous 24 month, which indicated congestion in the lungs but little to no enlargement of the heart. The medical record indicates that Patient B has had a history of acute complaints of shortness of breath and has experienced intermittent periods of persistent coughing. Recently, Patient B has experienced episodes of confusion and impaired thinking, which concerned him to the point where he contacted his primary care physician to discuss the episodes. During this conversation with his physician, Patient B also noted a feeling of being sick to his stomach, which the physician noted in his medical record. Patient B's history of high blood pressure, combined with the fact that he is a smoker with a previous history of episodic shortness of breath and persistent coughing make Patient B a potential heart failure candidate. His recent complaints of confusion and stomach problems are further indicia that trigger his categorization as a chronic heart failure patient candidate.

Thus, embodiments of a method and computer software product for identifying individual at risk for chronic heart failure indication in a population are disclosed to improve the accuracy of identifying potential chronic heart failure patients, decrease the time required to identify potential chronic heart failure patients so early intervention can be considered, identify potential chronic pain patients that meet the preference of stakeholders, and many other benefits. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method for identifying individuals at risk for chronic heart failure condition in a population, comprising:
selecting direct medical indicia associated with chronic heart failure that serve as independent variables;
selecting indirect medical indicia associated with chronic heart failure that serve as independent variables;
selecting non-medical indicia associated with chronic heart failure that serve as independent variables;
selecting a chronic heart failure indication that serves as a dependent variable;
creating a chronic heart failure model using direct medical indicia, indirect medical indicia, non-medical indicia, and chronic heart failure indication;
applying the chronic heart failure model to a population to create a patient mathematical expression for each member of the population; and,
identifying potential chronic heart failure patients by comparing each patient mathematical expression to selection objectives.

2. The method as in claim 1 wherein the chronic heart failure model comprises
a logic structure to define a logical decision process to operate on the independent variables and to progressively reach greater certainty about potential chronic heart failure patients;
weighted variables to reflect greater relevance of certain direct medical indicia, indirect medical indicia, and non-medical indicia to the chronic heart failure indication; and,
equations that represent relationships between or among weighted variables to form a chronic heart failure inference engine.

3. The method as in claim 2 wherein the chronic heart failure inference engine comprises,
at least fifty dependent variables;
at least thirty independent variables; and,
at least fifty equations.

4. The method as in claim 2 wherein the logic structure is developed using Chi-Square Automatic Interaction Detection (CHAID) analysis to establish relationships between a dependent variable and independent variables.

5. The method as in claim 2 wherein the logic structure is developed using Classification Adjusted Regression Tree (CART) analysis to establish relationships between the dependent variable and the independent variables.

6. The method as in claim 2 wherein the weighted variables are developed using logistical regression to establish relationships between the dependent variable and independent variables.

7. The method as in claim 2 wherein the weighted variables are developed using discriminate analysis to establish relationships between the dependent variable and independent variables.

8. The method as in claim 2 wherein appropriateness of patient indicia is evaluated using the Hosmer-Lemeshow Goodness of Fit Analysis.

9. The method as in claim 1 wherein the potential chronic heart failure patients are identified with a patient mathematical expression generated by the chronic heart failure inference engine operating on the patient indicia and the chronic heart failure indication.

10. The method as in claim 9 wherein the patient indicia are monitored and for changes and the patient mathematical expression is updated when patient indicia change.

11. The method as in claim 1 further comprising,
establishing categorization preferences that specify patient characteristics that are desired to be selected;
calculating the categorization preferences with each potential chronic heart failure patient's mathematical expression to identify relationships between the categorization preferences and each potential chronic heart failure patient's mathematical expression; and,
categorizing each potential chronic heart failure patient based upon the relationships between the categorization preferences and each potential chronic heart failure patient's mathematical expression.

12. The method as in claim 1 wherein the selection objectives are selected from the group consisting of potential chronic heart failure patients with heart failure attributable to their work environment, potential chronic heart failure patients unlikely to be compliant with treatment therapy, potential chronic heart failure patients unlikely to return to work, potential chronic heart failure patient suitable for low cost therapy, and potential chronic heart failure patient treatable by a primary care clinician.

13. The method as in claim 1 wherein the direct medical indicia are related to chronic heart failure in a known medical manner and recorded by a clinician.

14. The method as in claim 13 wherein the direct medical indicia are independent variables selected from the group consisting of primary diagnosis, associated secondary diagnosis, co-morbidities, drug treatment regimen, telephone consultations with a clinician, palliative care, rehabilitative care, clinician office visits, emergency room visits, and hospitalizations.

15. The method as in claim 13 wherein the sources for direct medical indicia are selected from the group consisting of claims records, medical records, workers' compensation records, and employer records.

16. The method as in claim 16 wherein indirect medical indicia are a chronic heart failure co-morbidity that is recorded by a clinician.

17. The method as in claim 1 wherein the indirect medical indicia are independent variables selected from the group consisting of mental health condition, acute respiratory episodes, diabetes, and hyperthyroidism.

18. The method as in claim 16 wherein the sources for indirect medical indicia are selected from the group consisting of claims records, medical records, workers' compensation records, employer records, and patient surveys.

19. The method as in claim 1 wherein the non-medical indicia are independent variables selected from the group consisting of alcohol consumption, smoking status, weight gain, life satisfaction measures, patient support structure, day-time distractions, marital relationship quality, personality profile, psychological profile.

20. The method as in claim 19 wherein the sources for non-medical indicia are selected from the group consisting of medical records, patient surveys, patient self-reports, employer databases, workers' compensation records, medical chart reviews, patient interviews, treating clinician interviews, and family member interviews.

21. The method as in claim 1 wherein the chronic heart failure indication is selected from the group consisting of congenital cardiovascular defects, structural heart defect, lung anomaly, uncontrolled blood pressure, and hyperthyroid.

22. The method as in claim 21 wherein the source for chronic heart failure indications is the International Association for the Study of Heart failure (IASP) chronic heart failure guidelines.

23. The method as in claim 1 wherein the patient population is selected from the group consisting of payer database, employer database, clinician database, and workers' compensation database.

24. A method for identifying and categorizing potential chronic heart failure patients, comprising:
accessing a chronic heart failure model having direct medical indicia, indirect medical indicia, non-medical indicia, and a chronic heart failure indication that are arranged logic structure, with weighted variables, and equations representing relationship between or among the variables;
applying the chronic heart failure model to a population to create a patient mathematical expression for each member of the population;
identifying potential chronic heart failure patients by comparing each patient mathematical expression to selection objectives;
establishing categorization preferences that specify characteristics of patents that are desired to be categorized;
calculating the categorization preferences with each potential chronic heart failure patient's mathematical expression to identify relationships between the categorization preferences and each potential chronic heart failure patient's mathematical expression;
categorizing each potential chronic heart failure patient based upon the relationships between the categorization preferences and each potential chronic heart failure patient's mathematical expression; and,
monitoring the potential chronic heart failure patient's direct medical indicia, indirect medical indicia, and non-medical indicia for changes and updating the patient's mathematical expression based upon changes to the potential chronic heart failure patient's direct medical indicia, indirect medical indicia, and non-medical indicia.

25. A computer software product that includes a medium readable by a computer, the medium having stored thereon instructions for identifying patients in a population having a chronic heart failure condition, comprising:
a first set of instructions when executed by the computer, causes the computer access a chronic heart failure model having direct medical indicia, indirect medical indicia, non-medical indicia, and a chronic heart failure indication that are arranged logic structure, with weighted variables, and equations representing relationship between or among the variables;
a second set of instructions when executed by the computer, causes the computer to applying the chronic heart failure model to a population to create a patient mathematical expression for each member of the population; and,
a third set of instructions when executed by the computer, cause the computer to identify potential chronic heart failure patients by comparing each patient mathematical expression to selection objectives.

26. The computer software product as in claim 25, further comprising,
a fourth set of instruction when executed by the computer, cause the computer to establish categorization preferences that specify characteristic of patents that are desired to be categorized;
a fifth set of instruction when executed by the computer, cause the computer to calculate the categorization preferences with each potential chronic heart failure patient's mathematical expression to identify relationships between the categorization preferences and each potential chronic heart failure patient's mathematical expression; and,
a sixth set of instruction when executed by the computer, cause the computer to categorize each potential chronic heart failure patient based upon the relationships between the categorization preferences and each potential chronic heart failure patient's mathematical expression.

27. A method for sensitivity analysis of a chronic heart failure patient model, comprising:
comparing the identified chronic heart failure patients with outside diagnosed chronic heart failure patient data to create a patient error list;
applying an error assessment model to the patient error list to identify the non-corresponding patient indicia that contributed to the errors;
applying a sensitivity analysis model to the non-corresponding patient indicia to identify potential patient indicia changes to reduce errors in identifying chronic heart failure patients;
selecting at least one patient indicia change from the potential patient indicia changes to apply to the patient indicia; and,
modifying the patient indicia with the at least one selected patient indicia change.

28. The method as in claim 27, further comprising
applying a sensitivity analysis model to the weighted variables to identify potential weighted variable changes to reduce errors in identifying chronic heart failure patients;
selecting at least weighted variable change from the potential weighted variable changes to apply to the weighted variables; and,
modifying weighed variables to reflect greater or lesser relevance of patient indicia to reduce errors in identifying chronic heart failure patients.
